(19) United States
(12) Patent Application Publication
(10) Pub. No.: US 2013/0183668 A1
(43) Pub. Date: Jul. 18, 2013

(54) METHODS RELATING TO IDENTIFICATION OF SUSCEPTIBILITY TO LIVER INJURY

(71) Applicants: Derek Mann, Newcastle Upon Tyne (GB); Jelena Mann, Newcastle Upon Tyne (GB); Mujdat Zeybal, Newcastle Upon Tyne (GB)

(72) Inventors: Derek Mann, Newcastle Upon Tyne (GB); Jelena Mann, Newcastle Upon Tyne (GB); Mujdat Zeybal, Newcastle Upon Tyne (GB)

(21) Appl. No.: 13/667,832

(22) Filed: Nov. 2, 2012

Related U.S. Application Data
(60) Provisional application No. 61/556,035, filed on Nov. 4, 2011.

(30) Foreign Application Priority Data
May 3, 2012 (GB) 1207788.9

(51) Int. Cl. C12Q 1/68

(52) U.S. Cl. CPC C12Q 1/6883
USPC 435/6.11

(57) ABSTRACT

The present invention relates to methods for identifying susceptibility of impaired hepatic wound healing in a patient, most particularly by identifying modifications of the of PPAR-γ and TGFβ1 genes. It further relates to stratifying populations of patients to determine susceptibility to impaired hepatic wound healing and direct appropriate healthcare resources. More specifically methods can be used to stratify liver disease patient populations to identify those most likely to progress to cirrhosis, or to identify the likelihood that a patient with liver disease will progress to having cirrhosis.

### CROSS-REFERENCE TO RELATED APPLICATIONS

The present non-provisional application claims priority to Great Britain Application 1207788.9, filed May 3, 2012, and PCT/GB2012/052714, filed Oct. 31, 2012 and the benefit of U.S. Provisional Application 61/556,035, filed Nov. 4, 2011, all of which are incorporated by reference herein in their entirety for all purposes.

### FIELD OF INVENTION

The present invention relates to methods for identifying susceptibility to impaired hepatic wound healing in a patient. It further relates to methods for stratifying populations of patients to determine susceptibility to impaired hepatic wound healing and to direct appropriate healthcare resources. More specifically, methods can be used to stratify liver disease patient populations to identify those most likely to progress to cirrhosis, i.e. to identify the likelihood that a patient with liver disease will progress to having cirrhosis.

### BACKGROUND OF THE INVENTION

Wound-healing is evolutionarily conserved but must be adaptive to new environmental challenges in order to maintain tissue homeostasis and prevent disease. The inventors have investigated the effect of ancestral liver damage on heritable reprogramming of wound-healing with surprising results. The inventors have discovered that male rats with a history of liver damage caused by chemical injury (in this case by carbon tetrachloride) transmit epigenetic adaptation of the wound-healing response to subsequent generations (F1 and F2) that serves to suppress fibrosis. Underlying this adaptation was increased expression of antifibrogenic PPARγ coupled with decreased expression of profibrogenic TGFβ1. Remodeling of both DNA (CpG) methylation and histone lysine acetylation underpinned these alterations in gene expression. Sperm isolated from rats with liver disease caused by either CCI4 or bile duct ligation (BDL) underwent histone modifications (H2A.Z and H3K27me3) at PPARγ that are associated with developmental annotation of DNA methylation. These histone modifications were also found in stem cells exposed to media conditioned by liver myofibroblasts suggestive of a mechanism for extra-hepatic transmission of epigenetic information. Most surprisingly of all, the inventors have determined that the initial findings in rats can also be seen in human populations. Non-alcoholic fatty liver disease patients with matched clinical parameters were stratified into two groups; severe liver fibrosis/cirrhosis and mild fibrosis. The latter group displayed hypomethylation of DNA at PPARγ gene promoter. These modifications paralleled those found in F2 generation rats protected from fibrosis as a consequence of multigenerational adaptation of hepatic wound-healing. Mechanisms therefore exist for heritable epigenetic reprogramming of wound-healing in response to environmental cues. The mechanisms appear to exist in a range of animals, more specifically mammals and most specifically and surprisingly humans allowing the inventors to develop methodologies for predicting susceptibility for progressing from liver disease to cirrhosis, in mammals, and specifically humans.

Wound-healing is a complex multi-step and multi-cellular process occurring in all metazoans that operates to restore tissue architecture and function following trauma or damage caused by environmental insults. During embryogenesis mammals repair wounds with remarkable efficiency and without scar (fibrosis) formation. By contrast, the ageing mammal is susceptible to either impaired repair in the form of chronic wounds, or to uncontrolled wound healing characterised by the progressive deposition of fibrotic tissue.

In humans, such deviations from optimal tissue repair underlie the development of life-threatening and age associated pathologies including cancers and tissue fibrosis. It is assumed that a combination of genetic and environmental factors dictate observed differences in the susceptibility of individuals to either impaired or uncontrolled wound-healing and their associated pathological consequences. However, the nature of the genetic traits and epigenetic mechanisms that impact on the efficiency of wound-healing and its outcomes remain poorly defined.

Liver cirrhosis is an example of a complex pathology that is highly variable within the patient population. The majority of patients with chronic liver disease (CLD) have pathologies with the potential to progress to cirrhosis, but only a minority (10-20%) of patients will ever go onto develop this life threatening end-stage disease. Mouse models suggest that genetic influences on disease progression in CLD are complex and likely to involve interactions of multiple low-impact variants. An alternative hypothesis is that exposure to environmental factors (including microbes and xenobiotics) may trigger adaptive epigenetic mechanisms that impact on the regulation of hepatic wound-healing. Adaptive epigenetic mechanisms may include alterations in DNA methylation, post translational modifications of histones and control of gene expression by non-coding RNAs. As these epigenetic signatures may become stable there is potential for non-genetic transmission of adaptive traits by both mitotic and meiotic routes, with the latter enabling intergenerational inheritance of epigenetically programmed traits. The inventors developed their work to test the hypothesis that population variability in hepatic wound-healing may be influenced by environmentally-induced adaptive traits transmitted between generations via heritable epigenetic signatures.

### SUMMARY OF THE INVENTION

The present invention identifies methods for testing patients for susceptibility to liver disease and for susceptibility to further progressing from chronic liver disease to cirrhosis. It also relates to potential therapies for liver disease and the production of animal models for studying the same.

According to a first aspect of the present invention, there is provided an in vitro method of determining susceptibility to liver disease in a patient (or susceptibility of impaired hepatic wound healing) comprising the steps;
providing a tissue sample obtained from a patient;
determining the level of (or amount of) DNA methylation of the PPAR-γ gene in the sample; and
using the level of DNA methylation to determine susceptibility to liver disease.

The amount of methylation can be determined as a percentage.

The method is carried out as an extracorporeal procedure.

Most preferably the method also comprises the step of comparing the DNA methylation level to predetermined threshold levels.

Most preferably the method also comprises the steps of; determining the level (or amount of) of DNA methylation of the TGFβ1 gene; comparing the DNA methylation level to predetermined threshold levels.

It has been shown that an increase in DNA methylation at the TGFβ1 gene and, concurrently, decreased DNA methylation at the PPAR-γ gene confers greater protection against liver disease. By comparing methylation levels in a sample to predetermined threshold levels it can be determined whether a patient has an elevated risk of progressing from early stage liver disease to cirrhosis.

Most preferably the step of determining the level of DNA methylation of the TGFβ1 gene determines the level of methylation of DNA at the TGFβ1 gene promoter.

Preferably the method of the determination of susceptibility to liver disease may be determining whether the patient is at a high risk of progressing to having cirrhosis.

Optionally, where the level of DNA methylation is less than 7% (more preferably lower than 6%) the patient is determined to have lower susceptibility to liver disease (an increased conferred protection).

Optionally, where the level of DNA methylation is greater than 7% the patient is determined to have higher risk of developing liver disease or for progressing from chronic liver disease to cirrhosis where the level of DNA methylation is 8-10% the patient may be considered high risk.

Optionally the method can be used to stratify groups of patients into groups susceptible to cirrhosis and those where liver disease is less likely to progress to cirrhosis.

Preferably the method determines levels of CpG methylation.

This can be expressed as a percentage.

Preferably, pyrosequencing is used to determine methylation levels.

Pyrosequencing is a method of DNA sequencing (determining the order of nucleotides in DNA) based on the “sequencing by synthesis” principle. Essentially, the method allows sequencing of a single strand of DNA by synthesizing the complementary strand along it, one base pair at a time, and detecting which base was actually added at each step. The template DNA is immobile, and solutions of A, C, G, and T nucleotides are sequentially added and removed from the reaction. Light is produced only when the nucleotide solution complements the first unpaired base of the template. The sequence of solutions which produce chemiluminescent signals allows the determination of the sequence of the template.

Alternatively DNA methylation-sensitive restriction enzyme mapping could be used to determine methylation levels.

Optionally the method also includes the steps of determining the amount of methylation of the PPAR-α gene and comparing said methylation to a predetermined threshold level.

Optionally the tissue sample is taken from the liver e.g. tissue from a biopsy.

Alternatively the tissue sample is blood or plasma.

The method may include the step of isolating DNA from the tissue sample. According to a further aspect of the present invention, there is provided an in vitro method of determining susceptibility to liver disease in a patient (or susceptibility of impaired hepatic wound healing) comprising the steps;
providing a tissue sample obtained from a patient;
determining the amount of one or more of PPAR-γ, and TGFβ1 proteins present in the sample; and using the amount of PPAR-γ, and TGFβ1 proteins present in the sample to determine susceptibility to liver disease.

Optionally the method also includes the step of comparing PPAR-γ, and TGFβ1 protein levels to predetermined threshold levels.

Optionally the method also includes the steps of determining the amount of PPAR-α protein and comparing said value to a predetermined threshold level value.

Optionally the tissue sample is taken from the liver e.g. tissue from a biopsy.

Alternatively the tissue sample is blood or plasma.

It has been shown that elevated expression of PPAR-γ and PPAR-α combined with decreased expression of TGFβ contribute to depressed transdifferentiation and is an indicator of greater protection against liver disease

According to another aspect of the present invention, there is provided an in vitro method of determining susceptibility to liver disease in a patient (or susceptibility of impaired hepatic wound healing) comprising the steps;
providing a tissue sample obtained from a patient;
determining the amount histone modification of a PPAR gene in the sample; and using the amount of histone modification of a PPAR gene in the sample to determine susceptibility to liver disease.

Optionally the method also includes the step of comparing the amount of histone modification to predetermined threshold levels.

Preferably the step of determining the amount histone modification of the PPAR gene identifies the amount of histone modification of the PPARγ gene.

Preferably the step of determining the amount histone modification of the PPARγ gene identifies the amount of H2A.Z and/or H3K27me3 present.

Preferably the step of determining the amount histone modification of the

PPARγ gene identifies the amount of histone modification of the PPARγ gene promoter.

Preferably the PPAR is a member of PPAR family comprising of PPARα, PPARβ/δ, and/or PPARγ.

Most preferably the member of PPAR family is PPARγ.

Preferably the TGF is a member of TGF family comprising of TGFα and/or TGFβ.

Most preferably the member of TGF family is TGFβ.

Optionally the tissue sample is taken from the liver e.g. tissue from a biopsy.

Alternatively the tissue sample is blood or plasma.

In order to better understand the invention various experimental work will now be described with reference to a number of figures which include;

### BRIEF DESCRIPTION OF THE FIGURES

FIG. 1—Presence of liver injury in male ancestors reduces liver fibrogenesis in F2 male offspring a) Adult outbred male rats were divided into vehicle control or a chronically injured liver group. Five rats from each group were harvested at peak fibrosis while another 5 from each group were bred following complete recovery from liver injury. This procedure was repeated with F1 generated males to produce F2 males which were then divided in groups A (no injury in F0 or F1), B (Injured F1, uninjured F0), C (injured F0, uninjured F1) and D (injury in both F0 and F1). Groups A-D were chronically injured and tissues collected at peak fibrosis (control uninjured group also included). b) Paraffin-embedded liver sections from injured rats in groups A-D were stained with Sirius Red (crosslinked collagen I and III). Panel in each row shows three separate animals from groups A to D. Scale bar, 100 μm. Right hand side panel is a representative H&E stain showing comparable level of liver injury in all groups. c) Percent area collagen I in each group was determined by automated scanning of 10 random fields in each slide using Leica QWin V3 (magnification 10×, n=5). d) Total RNA was isolated from livers and expression of collagen I mRNAs was quantified and normalized to β actin. Results are averages±s.e.m (n=5). Statistical analysis—one way parametric ANOVA, Tukey-Kramer post test. (*) p<0.05, (***) p<0.001.

FIG. 2—Paternally transmitted adaptation from fibrogenic response in chronic injury is mediated via a decrease in αSMA positive myofibroblasts in the liver. a) Paraffin-embedded liver sections from injured rats in groups A-D were stained with FITC labelled αSMA antibody. Panel in each row shows three separate animals from groups A to D. Scale bar, 100 μm. b) Average number of αSMA positive cells in each group was determined by manual counting of 10 random fields in each slide (magnification 20×, n=5). c) Total RNA was isolated from livers and expression of αSMA mRNAs was quantified and normalized to β actin. Results are averages±s.e.m (n=5). Statistical analysis—one way parametric ANOVA, Tukey-Kramer post test. (*) p<0.05, (***) p<0.001. d-e) Paraffin-embedded liver sections from control, uninjured (vehicle) groups A and D (in d) or injured rats in groups A and D (in e) were stained with anti desmin antibody. Panel in each row shows two representative animals. f) Average number of desmin positive cells in control or injured groups A and D was determined by manual counting of 10 random fields in each slide (magnification 20×, n=5). Statistical analysis—two-tailed Student's t-test. (**) p<0.01. g) Total RNA was isolated from livers and expression of TNFα mRNAs was quantified and normalized to β actin. Results are averages±s.e.m (n=5).

FIG. 3—Protected animals have altered expression of profibrogenic and antifibrogenic genes in their livers a) Expression of PPARγ, PPARα and TGFβ mRNAs was quantified and normalized to that of βactin. Results are averages±s.e.m (n=5). Statistical analysis—one way parametric ANOVA, Tukey-Kramer post test for A to D groups where (*) indicates p<0.05. b) Protein was extracted from group A and D livers; 30 μgs of total protein was separated by SDS-PAGE and immunoblotted for PPARγ and βactin. Gels shown are representative of two replicate experiments and show five animals in each group. c) Expression of TGFβ1 regulator Bambi and SMAD 1 to 7 mRNAs was quantified in livers from A and D group animals and normalized to βactin. Results are averages±s.e.m (n=5).) Results are averages±s.e.m (n=5). Statistical analysis—Student's t-test, (***) indicates p<0.001.

FIG. 4—Ancestral injury influenced fibrogenic response is limited to liver. a) Adult outbred male rats were divided into vehicle control or a chronically injured liver group. Five rats from each group bred following complete recovery from liver injury. Males produced from the breeding cycle (F1) were subjected to unilateral ureter obstruction (UUO) and fibrotic and control kidneys harvested 7 days after the surgery (n=10). b and c) Histological sections from obstructed kidneys in control and ancestral liver injury groups of rats were stained with Sirius red (b) or αSMA (c). d) Percent area αSMA in each group was determined by automated scanning of 10 random fields in each slide using Leica QWin V3 (magnification 100×, n=10). e and f) Total RNA was isolated from injured kidneys and expression of collagen I and αSMA mRNAs was quantified and normalized to βactin. Results are averages±s.e.m (n=10).

FIG. 5—Altered expression of profibrogenic and antifibrogenic genes in the livers of protected animals is underpinned by differences in DNA methylation. a) Genomic DNA was extracted from all livers in A and D rat groups; DNA was bisulfite treated and subjected to pyrosequencing using rat PPARα specific assay. Position of the differentially methylated CGs is shown in the schematic drawing above the graphs. Differences are expressed as percent DNA methylation (n=5). Statistical analysis—one way parametric ANOVA, Tukey-Kramer post test. (*) p<0.05, (**) p<0.01 and (***) p<0.001. b and c) genomic DNA as in a) was pyrosequenced using rat PPARα (b) or rat TGFβ1 (c) specific assay. Statistical analysis—one way parametric ANOVA, Tukey-Kramer post test. (*) p<0.05 and (***) p<0.001 for PPARα and two tailed Student's t-test, (**) indicates p<0.01, for TGFβ1. d) Crosslinked chromatin was isolated from livers of group A and D animals and ChIP assay carried out using anti acetylated histone H3 antibody. Region within rat PPARγ or TGFβ1 promoter were amplified from the ChIPed genomic DNA. Results were expressed as fold control IgG (n=5). Statistical analysis—Student's t-test, p=0.0159 (*) and p=0.0278 (*) for PPARγ and TGFβ1 respectively.

FIG. 6—Extrahepatic transmission of epigenetic modifications and evidence for modifications in DNA methylation at fibrogenic regulator gene associated with liver disease progression in humans. a-b) Male adult rats were given chronic CCI4 or olive oil (control) for 4 weeks, then recovered for 2 weeks. Mature sperm was isolated and crosslinked chromatin obtained which was used in ChIP assays with anti trimethylated histone H3 lysine 27 (H3K27me3) and anti H2A.Z. Using primers as outlined in FIG. 5d (region within PPARγ promoter) was amplified from the ChIPed genomic DNA. Results were expressed as fold control isotype matched antibody (n=5). b) Male adult rats underwent bile duct ligation (BDL) or sham operation (control); 15 days later mature sperm was isolated and crosslinked chromatin obtained which was used in ChIP assays with anti trimethylated histone H3 lysine 27 (H3K27me3) and anti H2A.Z (n=5). c) Rats were given CCI4 for 4 weeks and serum collected 48 h following last injection. Serum from injured or control animals was then injected intravenously into naive recipient rats twice a week for 4 weeks (using freshly collected serum each time). Sperm was harvested from serum recipient animals 24 h after the last injection of serum and ChIP assay followed by qPCR carried out as in a-b). d) Rat hepatic stellate cells were isolated and grown on plastic for 15 days; fresh media was applied then collected 48 h later. Rat bone marrow cells grown in MesenCult media to generate mesenchymal stem cells, which were treated with control or 48 h conditioned activated HSC media for 72 h. Crosslinked chromatin was then obtained and used in ChIP assays followed by qPCR as in a-b) (n=3). e) Human hepatic stellate cells were isolated and grown on plastic for 15 days and conditioned media collected. Human primary mesenchymal stem cells were treated with control or conditioned human activated HSC media for 72 h. Crosslinked chromatin was then obtained and used in ChIP assays followed by qPCR as in a-b). Primers were used that amplify a region within human PPARγ promoter. Results were expressed as fold control isotype matched antibody (n=3). f) NAFLD patient liver biopsy tissues were obtained from paraffin blocks, genomic DNA extracted, bisulfite treated and subjected to pyrosequencing using human PPARγ promoter specific assay. Position of the differentially methylated CGs is shown in the schematic drawing above the graphs. Statistical analysis—Mann Whitney test, where p=0.0013 for CpG1 and p=0.0047 for CpG2.

FIG. 7—analysis of patient data using a ROC curve

FIG. 8—

a) Rat TGFb1 genomic sequence (ENSRNOG00000020652) SEQ ID NO1

b) Rat PPARg genomic sequence (ENSRNOG00000008839) SEQ ID NO 2

c) Rat PPARa genomic sequence (ENSRNOG00000021463) SEQ ID NO 3

d) Human PPARg genomic sequence (ENSG00000132170) SEQ ID NO 4

FIG. 9—Histone modification data

a) Animals were injured with CCI₄ for 4 weeks, then allowed to recover for 2 weeks. Mature sperm was harvested and ChIP assay carried out for H3K27me3 and H2A.Z histone mofication/variant respectively. qPCR was done using rat TGFβ1 exon I specific primers. b) Rats were given CCI₄ for 4 weeks and serum collected 48 hours following last injection. Serum from injured or control animals was then injected IV into naive recipient rats twice a week for 4 weeks (using freshly collected serum each time). Sperm was harvested from recipient animals 24 hours after the last injection of serum and ChIP assay followed by qPCR carried out as in a).

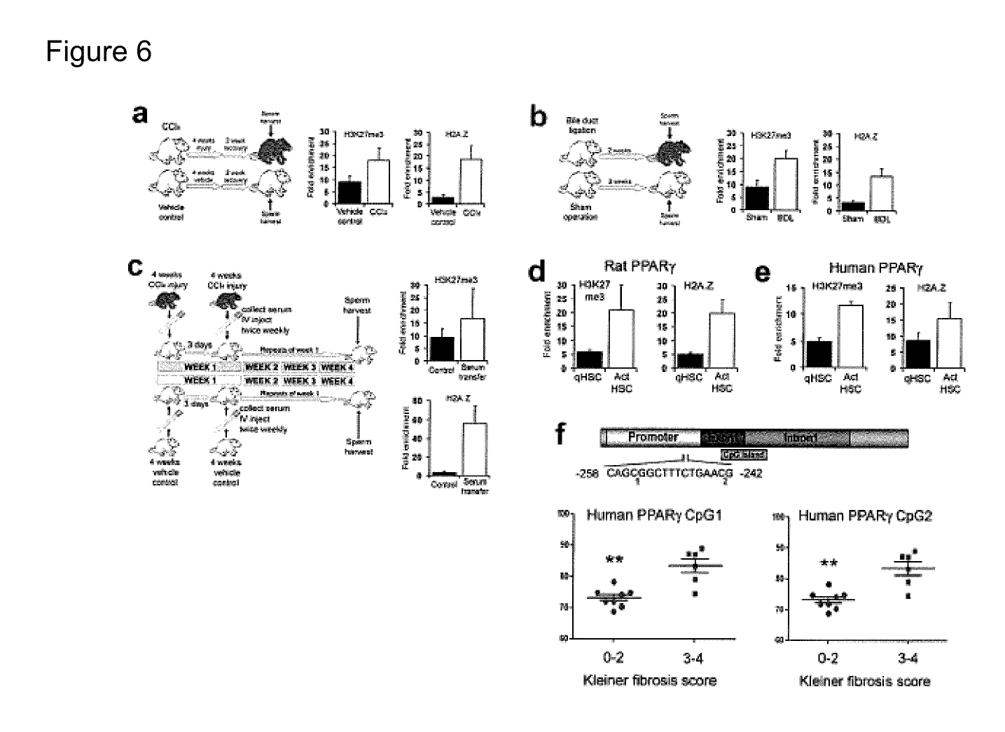

FIG. 10—baseline characteristics of patients used in FIG. 6f.

### DETAILED DESCRIPTION OF THE INVENTION

### Experimental Work

### Ancestral Liver Damage Promotes Multigenerational Adaptation of Hepatic Wound Healing.

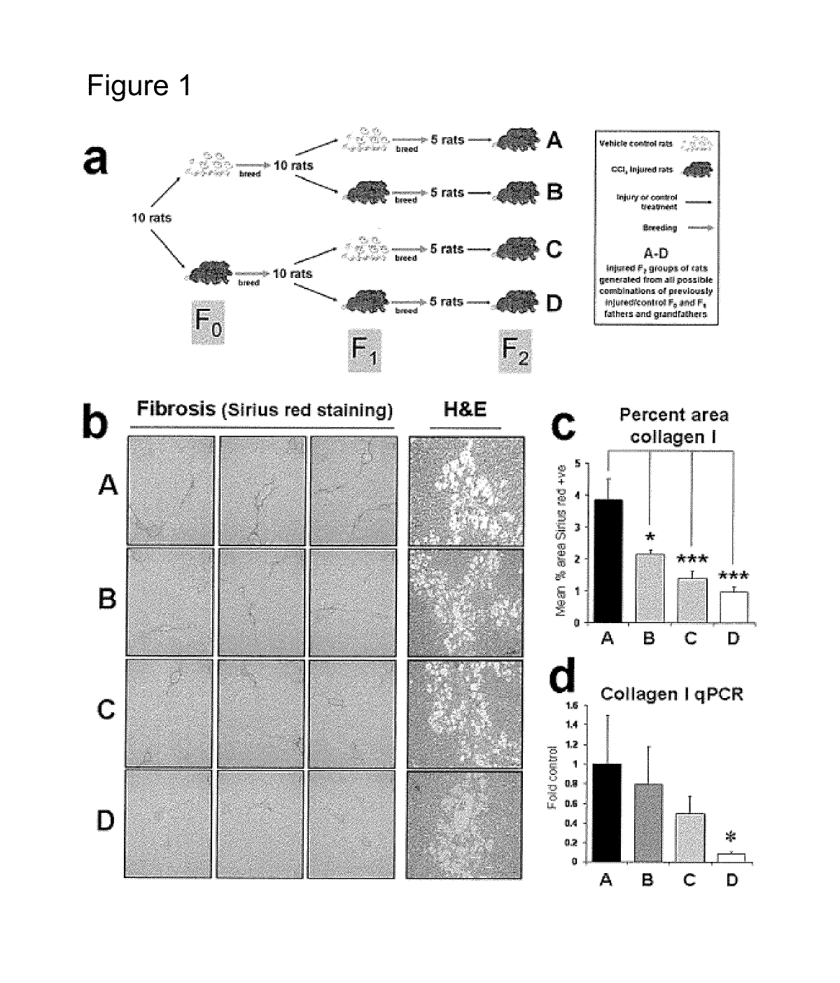

The inventors have developed a model for investigating multigenerational influences on liver fibrosis using outbred adult male rats (FIG. 1a). By selecting outbred animals the inventors mitigate against genetic traits impacting on wound-healing.

The rationale for studying transmission through the male line was to avoid influences from maternal factors either within somatic components of the oocyte or arising from the in utero environment, the latter being known to have major epigenetic influences on offspring. The model involved repeated injury of male rats by the hepatotoxin carbon tetrachloride (CCI4) to induce a state of chronic wound-healing resulting in fibrosis.

Cessation of injury allowed spontaneous resolution of fibrosis before the animals were paired with uninjured females for breeding. By repeating this process in the F1 offspring and with the inclusion of control sham (olive oil) injured animals we generated four groups of F2 males with distinct ancestral histories of liver disease (FIG. 1a). Group A had no history of injury in the F1 or F0 paternal lines. Group B was derived from injured F1 fathers and uninjured F0 grandfathers. Group C had a history of injury at F0 but not in the F1 generation. Group D were derived from male lines with injuries at both F1 and F0. All four adult F2 groups were then repeatedly injured with CCI4 and culled 24 hrs after the final CCI4 administration. Elevated serum liver enzymes (ALT and AST) were observed for all four groups with no significant differences between groups indicating similar degrees of hepatocyte damage.

From H&E stained sections (FIG. 1b) and pathology grading there were no obvious differences in necrosis or inflammation between groups. Cyp2E1, the major cytochrome p450 protein involved in metabolism of CCI4 to its reactive trichloromethyl metabolite, was expressed at similar levels between groups A and D as was Cyp3A4 which is also implicated in CCI4 metabolism. Therefore, ancestral liver injury and fibrosis has no heritable influence on CCI4 metabolism or its induction of hepatocellular damage.

To assess wound-healing, liver sections were stained with Sirius Red (SR) which selectively reacts with fibrotic (type I and III) collagens. Representative SR stained sections (FIG. 1b) illustrate for group A the expected network of bridging fibrotic septae. Morphometric analysis of SR stained sections revealed reduced levels of fibrotic matrix in groups B, C and D relative to A (FIG. 1c). For groups C and D, where fibrosis had been established at F0 generation, bridging fibrosis was rarely observed. Relative to group A, a modest reduction in hepatic procollagen I transcript expression was detected for group B, with 50% and 90% reduced expression observed for Groups C and D respectively, the latter reaching statistical significance (FIG. 1d).

It was concluded by the inventors that ancestral liver damage can trigger heritable adaptive mechanisms for suppression of the hepatic wound-healing response. Moreover, since for group C the disease trigger for this adaptation was in the F0 and not F1 it appears that this adaptive mechanism can be transmitted in a transgenerational as well as intergenerational manner.

### Adaptive Suppression of Hepatic Myofibroblast Activation.

Next the inventors determined the cellular basis for the influence of ancestral liver damage on wound healing.

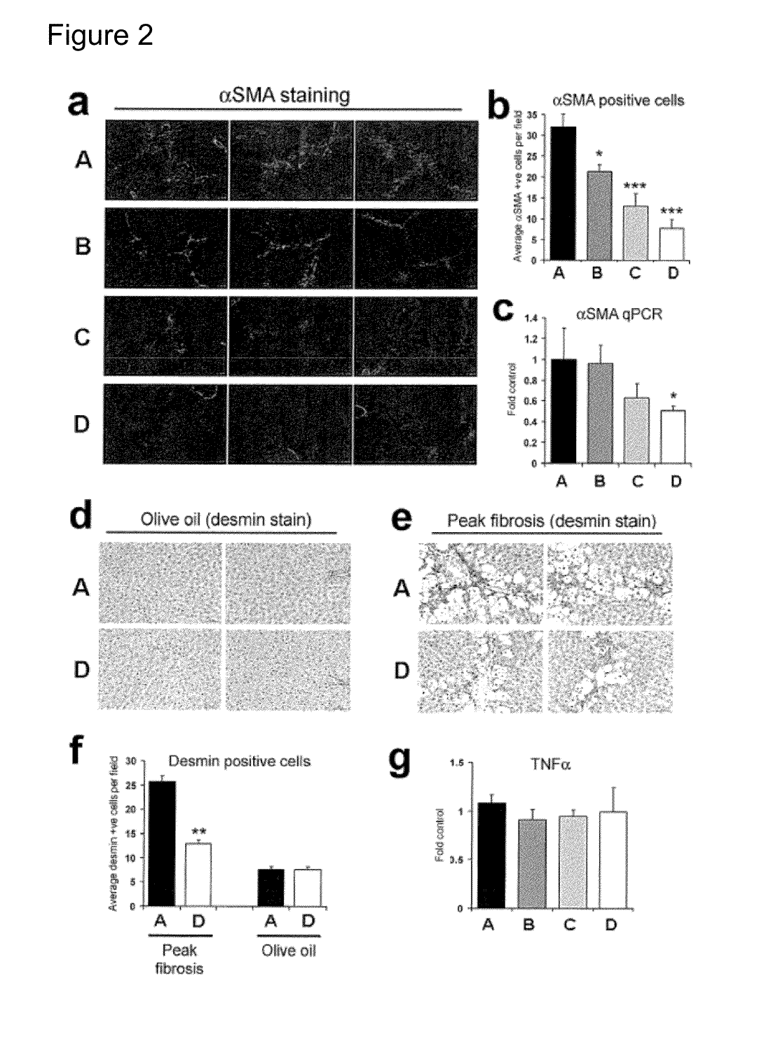

The major cellular drivers of liver fibrosis are smooth muscle α-actin (αSMA) positive myofibroblasts. Myofibroblasts are rare in the uninjured liver but accumulate in diseased liver mainly via transdifferentiation (activation) of hepatic stellate cells which adopt a highly proliferative and profibrogenic phenotype. αSMA positive myofibroblasts were observed within tracts of bridging fibrotic septae and in the proximity of necrotic, ballooning hepatocytes (dark patches in the stained tissue) (FIG. 2a). Manual counting of αSMA positive cells revealed reduced numbers of myofibroblasts in groups B, C and D relative to group A (FIG. 2b). Livers from animals in groups C and D had respectively 50% and 30% the number of myofibroblasts in group A livers, which was reflected by reduced abundance of hepatic αSMA mRNA (FIG. 2c). Possible explanations for lower numbers of myofibroblasts in groups B-D included a developmental reduction in numbers of hepatic stellate cells prior to injury or defects in the inflammatory response which is required for activation of hepatic stellate cells.

Desmin is a marker for both quiescent and activated hepatic stellate cells suitable for quantification of total numbers of these cells in normal and diseased liver. Sham injured group A and D livers contained similar numbers of desmin positive cells, i.e. approximately 8 cells/field (FIG. 2d and f). CCI4 injury increased these numbers to 26 and 13 desmin positive cells/field in group A and D respectively (FIG. 2e and f). These data argue against a developmental influence on numbers of hepatic stellate cells and instead suggest modulation of their activation is influenced by ancestral liver damage. As hepatic TNFα was expressed at similar levels in groups A to D it seems unlikely that there are gross differences in the hepatic inflammatory response (FIG. 2g). Moreover, manual counts of macrophages (CD68), T lymphocytes (CD3) and neutrophils did not indicate differences in inflammatory cell recruitment between all four groups of rats.

While these data do not completely exclude modulation of inflammation they are more supportive of an intrinsic reprogramming of the hepatic stellate cell phenotype that suppresses transdifferentiation.

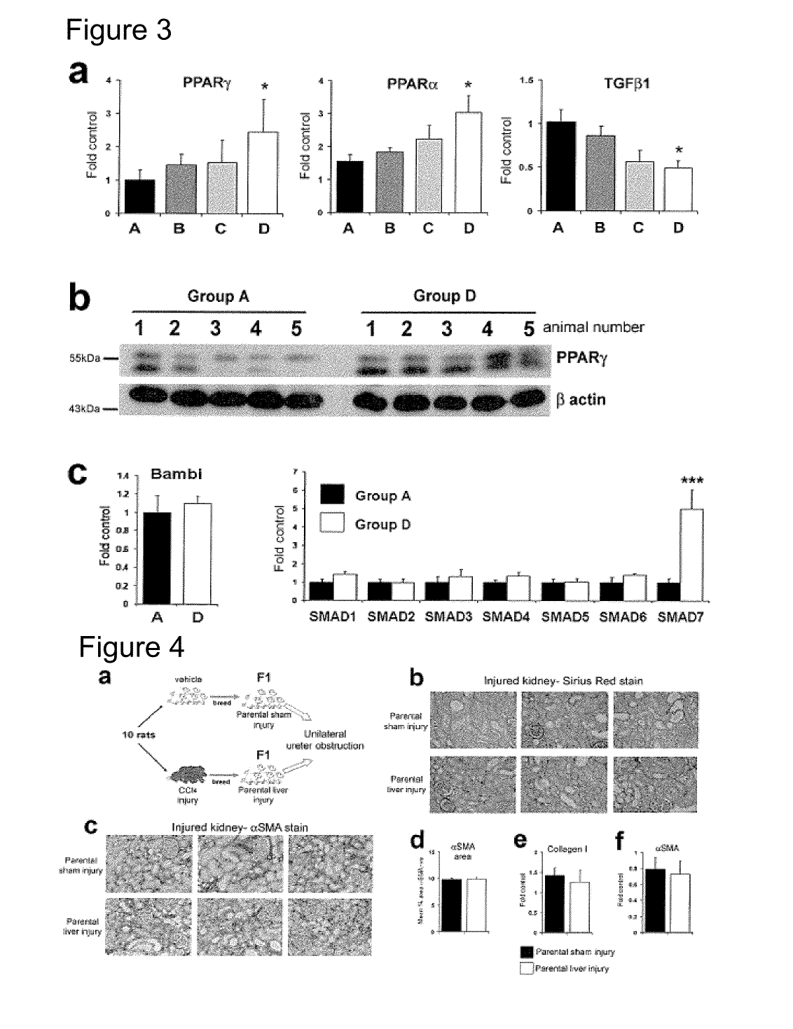

To begin to interrogate the molecular basis for modulation of hepatic stellate cell transdifferentiation the inventors selected candidate genes based on their role in wound-healing. Quiescent hepatic stellate cells have an adipogenic-like phenotype controlled in part by nuclear receptors PPAR-γ and PPAR-α which are repressed during transdifferentiation. PPAR-γ expression is repressed by DNA-methylation and MeCP2-dependent chromatin remodelling that is critical for generation of the myofibroblast phenotype. Hepatic expression of PPAR-γ was elevated in groups B, C and D compared with A (FIG. 3a), and immunoblotting confirmed raised levels of PPAR-γ protein in group D vs A livers (FIG. 3b). This differential expression of PPAR-γ is relevant since ectopic re-expression in myofibroblasts can reverse their phenotype into a more quiescent state. Additionally, we observed increased expression of PPAR-α in group C and D (FIG. 3a). In contrast hepatic TGFβ1, a highly profibrogenic factor induced during hepatic stellate cell activation, was reduced 2-fold in group D compared to A and trends towards reduced TGFβ1 expression also noted in groups B and C (FIG. 3a). By contrast, there were no differences in expression of the TGFβ pseudoreceptor Bambi or the TGFβ1 signalling molecules SMADs 1-6, whereas inhibitory SMAD7 was elevated 5-fold in group D compared with A (FIG. 3c). cDNA array analysis identified additional genes displaying differential expression between groups A and D. Of those genes that were either significantly over- or underexpressed in group D several have been implicated as regulators of fibrogenesis and/or hepatic stellate cell activation including Matrix Metalloproteinase 3 (MMP3=+3.3 fold), IGF Binding Protein 1 (IGFBP1=+2.8 fold), Secreted Phosphoprotein 1 (SPP1=+2.5 fold), One Cut homeobox 1/HNF6 (ONECUT1=−4.8 fold) and Suppressor of Cytokine Signaling 2 (SOCS2=−2.4 fold).

The inventors conclude that liver damage has an intergenerational influence on the transcriptional control of multiple genes in hepatic stellate cells and that elevated expression of PPAR-γ and PPAR-α combined with decreased expression of TGFβ contribute to depressed transdifferentiation.

### Renal Fibrosis is not Influenced by Ancestral Liver Damage.

A question that arose from the inventor's observations was whether fibrosis in the liver has a global impact on wound-healing in future generations. To address this the inventors generated F1 adult male rats that either had history of CCI4-induced parental liver fibrosis (PLF) or control parental sham injury (PSI) (FIG. 4a). Renal injury was then induced in PLF and PSI groups using the unilateral ureteral obstruction (UUO) model. Histological SR (FIG. 4b) and αSMA (FIG. 4c) stains revealed no differences in collagen deposition or myofibroblast accumulation respectively between the two groups of animals. Since the PLF group historically resemble group B from the multigenerational model in that these animals will have inherited a suppressed hepatic wound-healing response associated with reduced expression of collagen I (FIGS. 1c and 1d) and αSMA (FIG. 2b and c). The inventors concluded that intergenerational adaptation to liver injury must be selectively targeted at mechanisms regulating hepatic wound-healing rather than reflecting a global impact on wound-healing process in other organs.

### Epigenetic Modifications at Gene Loci Regulating Hepatic Myofibroblast Activation.

The inventors investigated a potential epigenetic basis for altered PPARγ and TGFβ1 expression initially focusing on DNA methylation which generally acts to repress gene transcription.

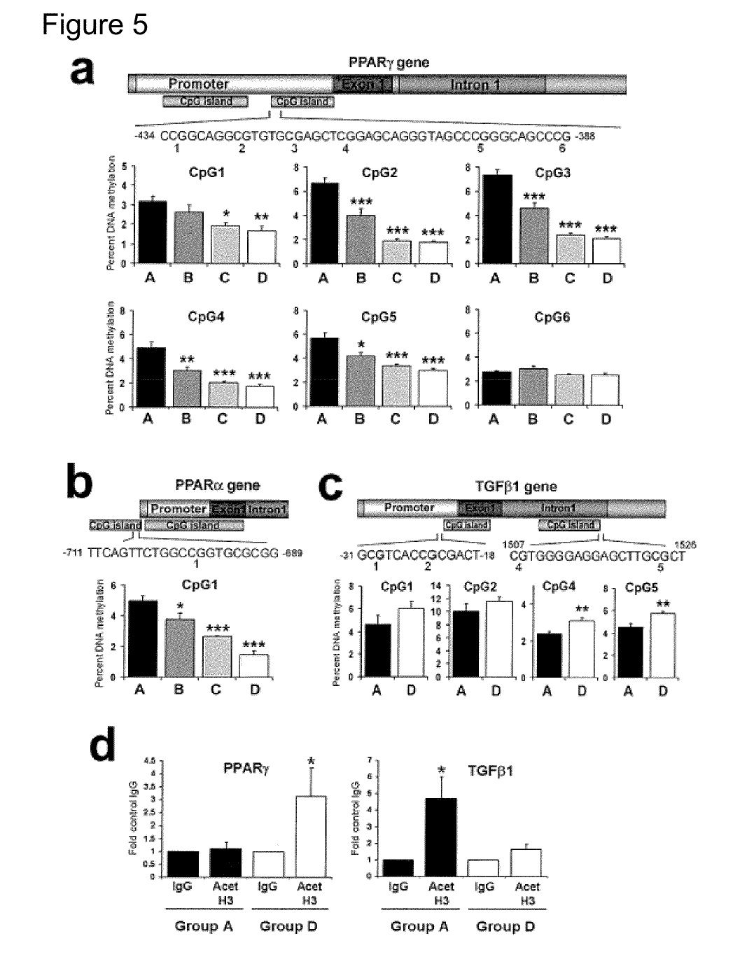

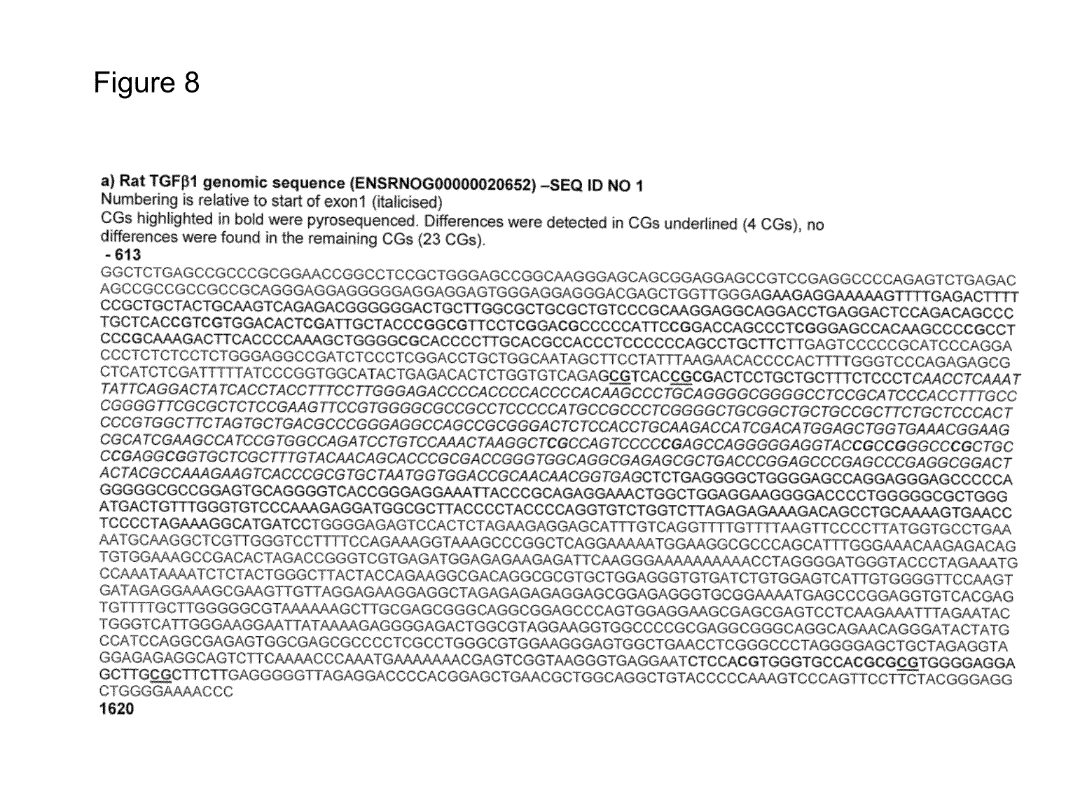

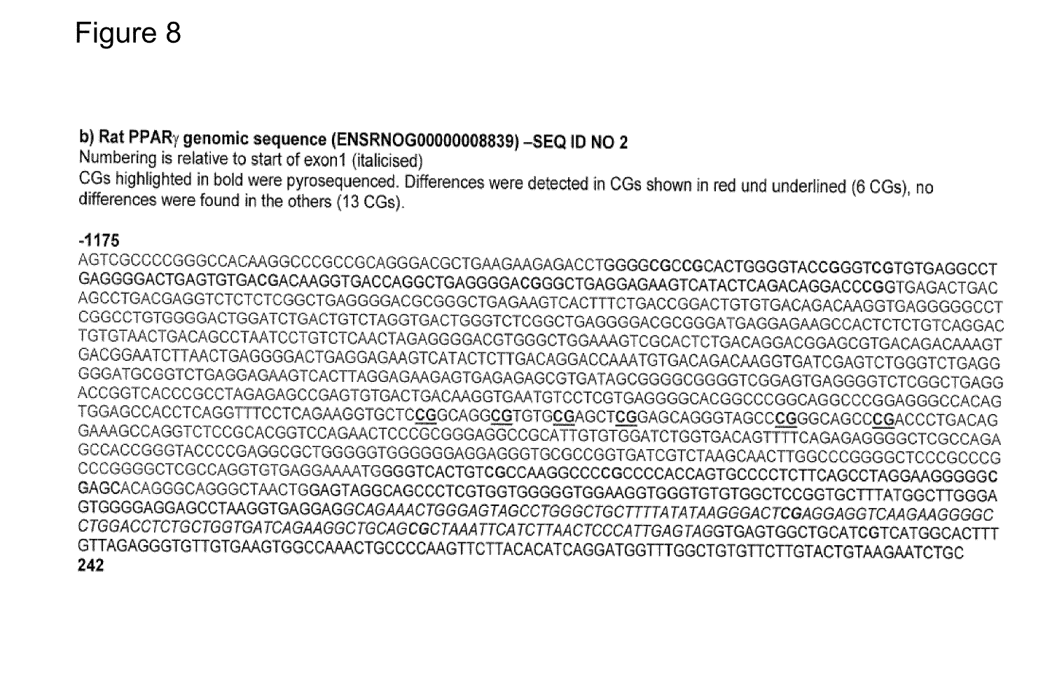

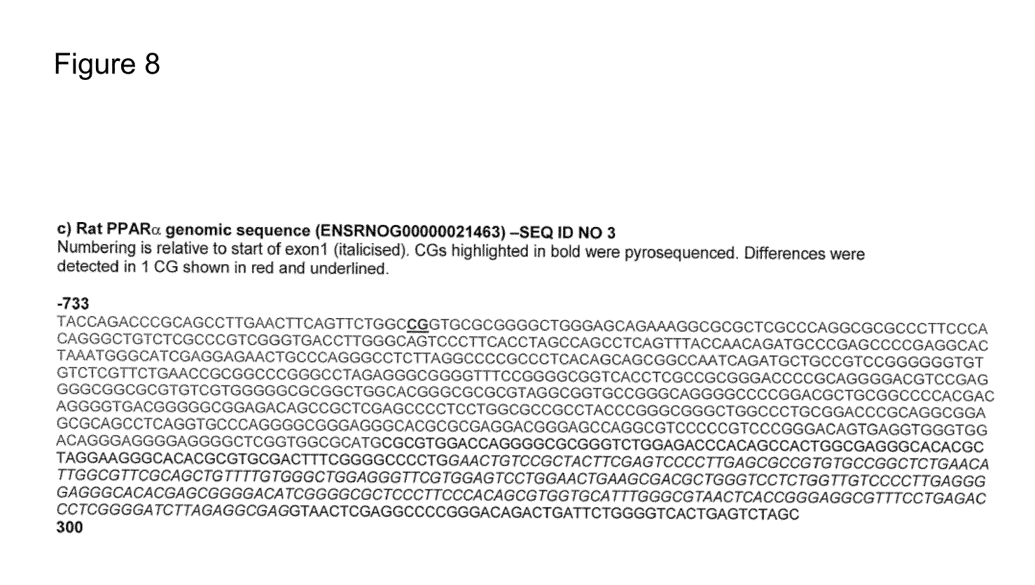

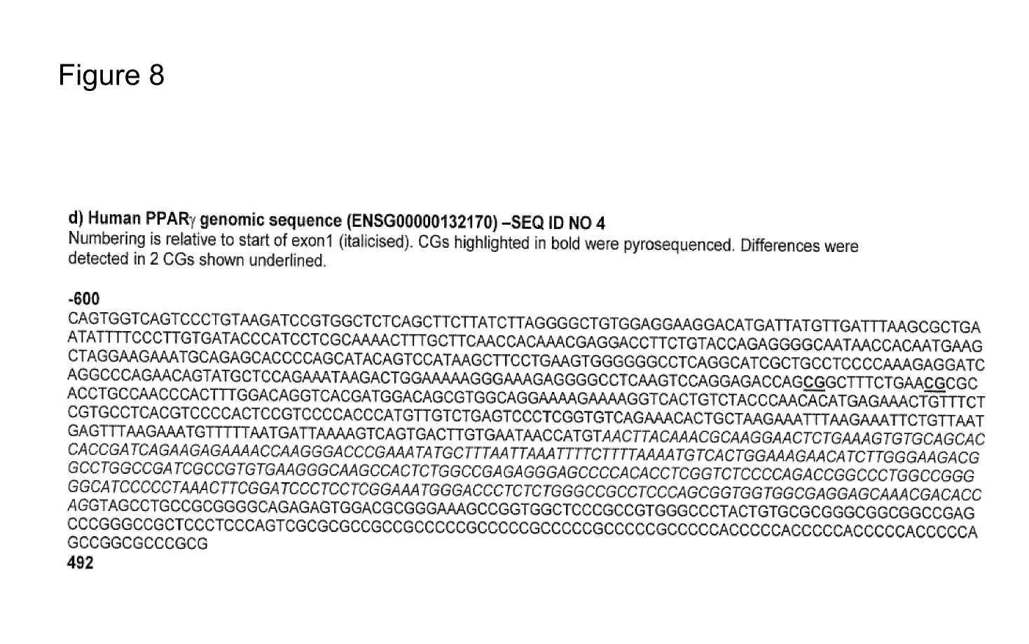

To refine the methylation profiling and identify differences in methylation at specific CpG sites we developed quantitative sequence-specific CpG methylation (pyrosequencing) assays for the rat PPAR-γ, PPAR-α and TGFβ1 genes (FIGS. 8a-c). Pyrosequencing across the PPAR-γ promoter identified altered methylation at five consecutive CpG sites within a 20 bp sequence (FIG. 5a). At the three central sites in this sequence (CpG2, CpG3 and CpG4) reduced methylation was found in groups B, C and D. In each case group D was associated with the lowest level of methylation at these sequences, in the case of the most central CpG sequence (CpG3) we detected 7% methylation in group A compared with 2% in group D. Similar analysis of the PPAR-α promoter identified a single CpG site which was hypomethylated in groups B, C and D (FIG. 5b). Pyrosequencing of the TGFβ1 gene identified four CpG sites displaying modest increases in methylation which for two adjacent CpG sites in the intron 1 region reached statistical significance (FIG. 5c).

One way in which DNA methylation may regulate gene transcription is by modelling basal states of histone acetylation which impact on the accessibility of chromatin for transcription factors. Quantitative chromatin immunoprecipitation (qChiP) assays revealed higher enrichment of AcetH3 at the PPAR-γ promoter in group D compared with group A, whereas AcetH3 at the TGFβ1 promoter was reduced in group D relative to A (FIG. 5d). Hence the observed adaptive changes in expression of fibrogenic regulators are underpinned by remodeling of DNA methylation and associated histone acetylation such that antifibrogenic PPAR-γ becomes more transcriptionally permissive whereas profibrogenic TGFβ becomes less permissive for transcription.

Chromatin Modifications in Sperm Correlate with Intergenerational Adaptation of Wound-Healing.

The inventors investigated the mechanisms by which the damaged liver may exert an intergenerational influence on hepatic stellate cell gene expression and wound-healing.

DNA methylation is unlikely to serve this purpose directly as there are at least two episodes of genome-wide remodelling of DNA methylation occurring during zygote and embryonic development.

Therefore although ancestral tissue damage is associated with altered hepatic DNA methylation in the F2 liver tissue, these differences must be annotated somehow during embryo development. There is emerging data that specific histone signatures can influence DNA methylation at developmentally regulated genes. The highly conserved variant histone H2A.Z appears to be mutually exclusive with DNA methylation and its incorporation into nucleosomes may suppress CpG methylation during embryo development. Genome-wide studies in embryonic stem cells reveal that H2A.Z is enriched at the promoters of genes functioning in cell differentiation and development.

Interestingly the majority of genes occupied by H2A.Z are targets for the transcriptional repressor Polycomb proteins including the PRC2 proteins that regulate H3K27 trimethylation (H3K27me3). Since the inventors had determined previously that PPAR-γ is a Polycomb regulated gene they were intrigued to determine whether H2A.Z is incorporated into chromatin at the PPAR-γ promoter in sperm. Of note, although the majority of histones are replaced with protamines during development of sperm, a significant proportion of the genome of mature sperm remains associated with histones including variant H2A.Z. Sperm were isolated from male rats that had been injured with CCI4 or vehicle control for 4 weeks and then allowed to recover for 2 weeks, during which time wound-healing fully resolves. Crosslinked qChIP was then used to quantify incorporation of H2A.Z and H3K27me3 into chromatin at the PPAR-γ promoter (FIG. 6a).

Both histone signatures were associated with the PPAR-γ promoter and were found at increased levels from sperm of rats recovering from fibrosis relative to controls. H3K27me3 was only modestly enriched, whereas H2A.Z was found at 6-fold higher levels suggesting that the variant histone is incorporated into PPAR-γ chromatin as a consequence of liver damage.

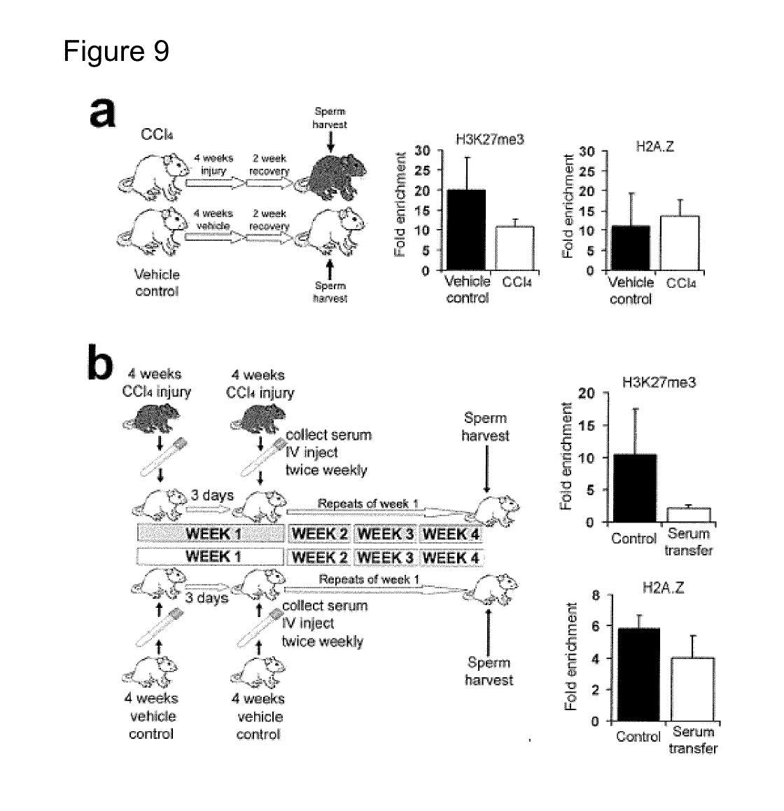

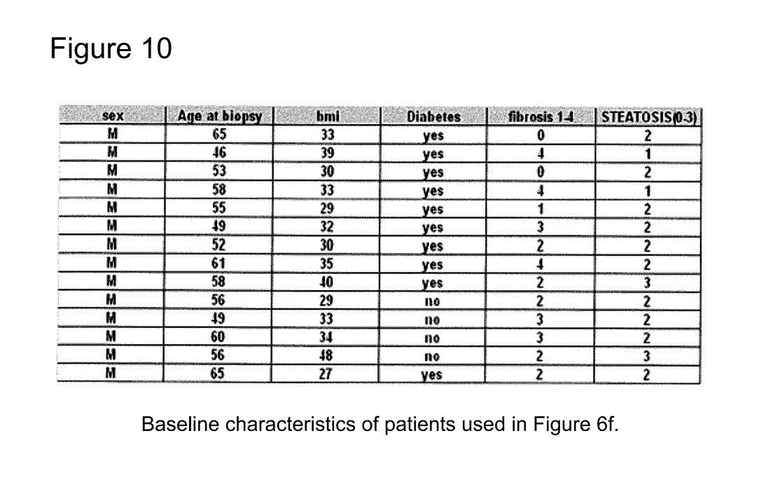

To confirm this observation, the inventors carried out an identical analysis in chromatin prepared from the sperm of rats injured by bile duct ligation (BDL), a surgical liver injury model which causes severe tissue damage and fibrosis but by injury mechanisms which are distinct from the CCI4 model. The inventors again observed a modest enrichment of H3K27me3 and a substantive 4-fold increased incorporation of H2A.Z (FIG. 6b). From these data we suspected that liver damage results in the accumulation of a soluble factor in serum that can modify chromatin structure either in germ stem cells or/and mature sperm. To test this idea we repeated the qChIP analysis of histone signatures in sperm from uninjured rats that had undergone repeated serum transfers from rats that had been injured with CCI4 for 4 weeks prior to a 48 h recovery to ensure clearance of the chemical and its active metabolites (FIG. 6c). Remarkably, serum transfer induced a modest increase in PPAR-γ-associated H3K27me3 and a 15-fold enrichment of H2A.Z. ChIP assays at the TGFβ1 gene did not demonstrate alterations in chromatin associated H2A.Z but detected reduced H3K27me3 both in sperm from CCI4 injured rats and sperm from animals in the serum transfer experiment (FIG. 9).

The inventors asked if hepatic stellate cell-derived myofibroblasts may be a source of the soluble mediator of PPAR-γ chromatin remodelling. Isolated primary hepatic stellate cells will undergo myofibroblast transdifferention when cultured on plastic and in full media. We therefore collected media conditioned by cultured rat hepatic stellate cells and added this to cultures of rat bone marrow-derived mesenchymal stem cells (rMSCs). ChIP analysis showed a 3-fold enrichment of H3K27me3 and a 4-fold increase in recruitment of H2A.Z to the PPAR-γ promoter (FIG. 6d). To determine the relevance of these data for human liver disease we next collected conditioned media from cultures of primary human hepatic stellate cells and exposed three independently prepared and phenotyped human MSCs (hMSCs) to the media (FIG. 6e. We again observed increased association of H3K27me3 and H2A.Z with the PPAR-γ chromatin in hMSC exposed to conditioned media compared with control cultures.

Finally, the inventors asked if differences in PPAR-γ promoter methylation can be observed between human livers with mild (Kleiner score 0-2) or severe (Kleiner score 3-4) fibrosis (Kleiner scoring being a histological scoring system for nonalcoholic fatty liver disease based on relatively simple, objective factors).

DNA was prepared in a blinded manner from archived liver biopsy samples from a well characterised males between the ages of 46 to 65 with biopsy proven non-alcoholic fatty liver disease (NAFLD) (for details see FIG. 10). Pyrosequencing targeting two me-CpG sites in the human PPAR-γ promoter demonstrated hypermethylation (approx 10% higher at both dinucleotides) associated with severe versus mild fibrosis (FIG. 6f). These latter data lend support to the concept that wound-healing in the human liver may be influenced by stable epigenetic signatures.

More particularly the inventors determined whether the changes identified in their initial work occur in patient Non-Alcoholic Steatohepatitis (NASH) cohort. They obtained 15 biopsies from NASH patients with clearly defined demographics such that they were age, sex and diabetes status matched but could be stratified into patients that had none/mild fibrosis versus those with moderate/severe fibrosis. Based on their fibrosis score, these patients were termed non-fibrotics (NF) or fibrotics (F).

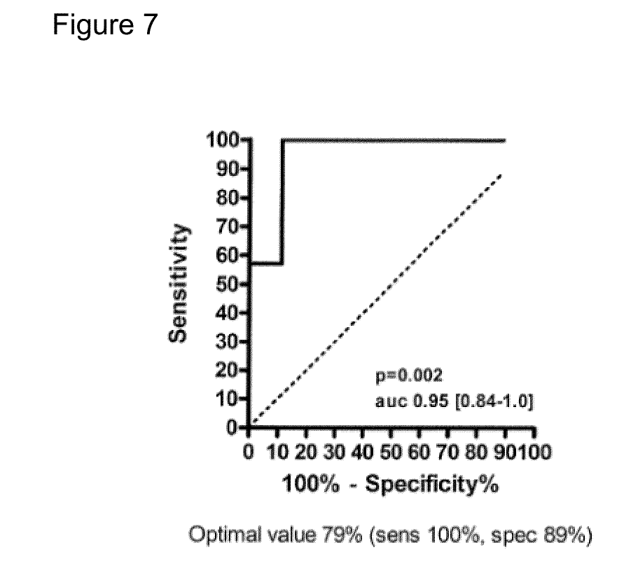

The inventors also analysed the patient data using a ROC curve (shown in FIG. 7). The ROC curve is a fundamental tool for diagnostic test evaluation. In a ROC curve the true positive rate (Sensitivity) is plotted in function of the false positive rate (100-Specificity) for different cut-off points of a parameter. Each point on the ROC curve represents a sensitivity/specificity pair corresponding to a particular decision threshold. The area under the ROC curve is a measure of how well a parameter can distinguish between two diagnostic groups (diseased/normal). In short, this test tells us how valuable a test is as a clinical tool and identifies an optimal cut-off. In this case the patient data shows that diagnostic tests to stratify patients or to indicate patient susceptibility to liver disease and cirrhosis would be both highly specific and sensitive and therefore of significant value.

### Discussion

The idea that epigenetic signatures can be inherited in a intergenerational fashion is supported by recent experimental studies. Alterations in germ cell DNA methylation caused by in utero exposure to vinclozolin mediate transgenerational transmission of adult-onset pathologies in multiple organs. Ng et al recently reported recently that feeding adult male rats a high fat diet led to insulin-resistance in female offspring associated with alterations in the expression of 642 β-cell genes. Feeding a low protein diet to male mice altered global CpG methylation patterns in offspring associated with adaptation of hepatic lipid and cholesterol metabolism.

Here the inventors extend and advance these studies by demonstrating for the first time that hepatic wound-healing can be subject to intergenerational adaptation, such that an ancestral history of fibrosis suppresses fibrogenesis in future generations. This adaptation was transmitted both between successive and across three generations indicative of multigenerational influence.

Reduced fibrogenesis was apparent within a single generation but appeared to be more pronounced when transmitted across generations and particularly where fibrosis had been present in successive F0 and F1 animals suggestive of a cumulative process. It is tempting to speculate on what might be the biological purpose of this intergenerational adaptation. The liver has a remarkable intrinsic capacity to rapidly and repeatedly regenerate any lost or damaged parenchymal tissue to maintain its function. As such it can be argued that deposition of fibrotic tissue can only be detrimental to liver repair while the organ retains its regenerative ability, which in the case of rodents and humans is maintained during the reproductive years. Hence, intergenerational down-regulation of liver fibrogenesis may have the biological advantage of ensuring greater fitness of future generations faced with the environmental pressures of liver toxins. Such adaptation would be potentially disadvantageous if it led to diminished fibrogenesis in other organs that lack the regenerative capacity of the liver. However a key discovery in our studies was that ancestral liver fibrosis did not appear to alter the development of fibrosis in the injured kidney. As yet the inventors have not further elucidated the mechanistic basis for this apparent organ-specific adaptative response in fibrogenesis, although clues may eventually emerge from further investigations of the epigenetic processes underlying its intergenerational transmission.

PPAR-γ is an important transcriptional repressor of the myofibroblastic phenotype of activated hepatic stellate cells that is down-regulated at the transcriptional level during the initiation phase of transdifferentiation. Experimental modulation of PPAR-γ expression or treatment with PPAR-γ agonists acts to suppress the fibrogenic features of hepatic stellate cells and in vivo blunts the development of liver fibrosis. Our discovery that PPAR-γ and the functionally related gene PPAR-α are both over-expressed in the liver of rats with an ancestral history of fibrosis is therefore highly relevant and at least in-part provides a molecular basis for reduced hepatic stellate cell activation in these animals. Hypomethylation at specific CpG residues within regulatory regions of the PPAR-γ and PPAR-α and associated enrichment of Histone 3 acetylation helps explain elevated hepatic expression of these genes, however since the same CpG methylation signatures were not found in the spleen of rats with a history of fibrosis they appear be tissue/cell-restricted marks that are unlikely to provide a mechanism for intergenerational transmission. Instead, we propose that an as yet uncharacterised soluble factor released by hepatic stellate cells is able to stimulate chromatin remodelling at regulatory genes in stem cells including germ cells and/or mature sperm. For PPAR-γ we observed H2A.Z incorporation into chromatin of sperm either in response to liver fibrosis or serum transfer from fibrotic animals. H2A.Z is of particular interest in this context since it provides mechanisms for so-called “epigenetic memory”. H2A.Z is enriched at the promoters of transcriptionally repressed genes and is more easily removed than H2A enabling more rapid re-activation of transcription. Furthermore, H2A.Z containing nucleosomes prevent the spread of heterochromatin and can be an antagonistic signature for DNA methylation, suggesting that H2A.Z has important anti-silencing functions.

Incorporation of H2A.Z into the PPAR-γ chromatin within sperm in response to liver damage may therefore influence either the annotation of PPAR-γ DNA methylation during embryonic development or the structural organisation of the PPAR-γ gene within chromatin in a manner that impacts on its potential for transcription. While our chromatin studies were restricted to PPAR-γ the identification of altered DNA methylation status in the PPAR-α and TGFβ1 genes indicates that heritable chromatin signatures most likely assemble at a variety of genes in sperm and combine to adaptively remodel the epigenetic landscape of the resulting hepatic stellate cell.

Additional unanswered questions arising include the identity and nature of the soluble mediator(s) of epigenetic remodeling in stem cells and sperm. To answer the former question it will be necessary to systematically interrogate the functions of the plethora of bioactive molecules secreted by hepatic stellate cells which include hormones, growth factors, cytokines, morphogens and proteolytic enzymes. To determine if adaptive signatures within the epigenome can influence fibrosis progression it will be necessary to comprehensively identify these signatures and then carry out detailed prospective clinical studies that ask if they correlate with disease outcome.

### Methods Summary

The chronic CCI4 injury was carried out for 4 weeks as previously described. Rats were either sacrificed 1 day after the last CCI4 treatment, or allowed to recover for 2 weeks after which time they were bred with females. Male offspring was mixed so that father from each treatment group had a male offspring in each of the relevant group in the next generation thus avoiding the possibility of genetic influence over phenotype—five fathers from olive oil treatment group in F0 had a representative male offspring each in the olive oil and CCI4 treatment groups in F1 and so on. Liver sections from all animals were cut and stained with Sirius Red, hematoxylin and eosin or FITC conjugated antibody targeting SMA as previously described. Desmin, macrophage, lymphocyte and neutrophil staining were carried out using anti-desmin antibody D33 (Abcam-ab8470), mouse anti rat CD68 (Serotec-MCA341R), rat anti human CD3 (Serotec-MCA1477) and Naphthol AS-D Chloroacetate (Specific Esterase) Kit (Sigma-Aldrich). Percent area collagen I was determined using Leica QWin V3 imaging software. Necrosis and inflammation were scored using a scale. The scale for necrosis was 0=none, 1=focal ballooning and/or hepatocyte necrosis, 2=confluent necrosis/ballooning. The scale for inflammation was 0=none, 1=occasional inflammatory cells, 2=focal collections of inflammatory cells, 3=extensive inflammatory infiltrate. qPCR was carried out as published previously with the primers listed in Supplementary Table 1. Western blot was done as previously described using 1 μg/ml anti PPARγ antibody (Abcam, ab19481), anti-cytochrome P450 2E1 antibody-(Abcam, ab28146), anti-cytochrome P450 3A4 antibody (Abcam, ab3572).

Bile duct ligation/sham was carried out as previously described. Mature sperm was isolated by flushing out the vas deferens with warm PBS as well as allowing sperm to swim out of a singly cut epididymis. Chromatin was made from fresh sperm using standard crosslinking method (3 minutes at room temperature) and sonication in Diagenode water bath sonicator (10 cycles of 30 s on, 20 s off at high power). Antibodies used in ChIP were raised against H3K27me3 (Diagenode, pAb-069-050), H2A.Z (Abcam, ab4174) or ChIP control antibody (Abcam). Liver tissue ChIP was carried out as in; sperm ChIP was modified as outlined in FIG. 6 legend.

The promoter regions of the genes were estimated using Genomatix

Gene2 promoter (Genomatix Software Gmbh, Ann Arbor, Mich., USA). CpG islands were determined by EMBOSS CpG Plot tool (http://www.ebi.ac.uk/Tools/emboss/cpgplot/).

Genomic DNA was extracted from the rat livers using phenol-chloroform extraction method. Retrospective non-alcoholic fatty liver disease (NAFLD) patient samples were obtained from archives of local Health Trust under a current ethical approval (approval number 06/Q0905/150). Paraffin embedded patient liver biopsy tissue was dewaxed and genomic DNA obtained. 2 μg of genomic DNA were bisulfite treated by EZ DNA methylation kit (Zymo Research, Irvine Calif.). Bisulfite modified DNA was amplified by primers either described in table 1 or by Rn-Ppparg-03 and Rn-Ppara-02 PM PyroMark CpG assay (PM00549535, PM00581903 Qiagen). 10 μl of PCR product was used in pyrosequencing reaction. The percentage methylation at each CpG sites was measured by Pyromark Q96 MD (Qiagen) and analysed by PyroQ-CpG™ 1.0.6 software (Biotage, Uppsala, Sweden).

It is envisaged that patients can be stratified into groups indicating their susceptibility to liver disease and likelihood of liver disease progressing to cirrhosis by determining the percentage of methylation by looking at results from known groups. From current results methylation of PPARγ of less than 7% suggests lower susceptibility (greater conferred protection) less than 5% suggests lower susceptibility and less than 2% very low susceptibility. Methylation of greater than 7% suggests higher susceptibility to liver disease with 10% or higher being indicative of significant risk confers lower protection. However, the % against which lower and higher susceptibility is graded may be changed.

Primary human and rat hepatic stellate cells (HSC) were generated as before. Rat mesenchymal stem cells (MSC) were obtained by culturing isolated bone marrow from rat femurs in MesenCult medium (StemCell technologies) for 2 weeks when they were cultured for 72 h with conditioned media collected from 48 h culture of activated rat HSCs. Human bone marrow stem cells were isolated from human bone marrow mononuclear cells (Lonza Biosciences, Berkshire, U.K.). Cells were grown and expanded in monolayer culture in Mesenchymal Stem Cell Growth Medium Bullet Kit (Lonza) supplemented with 5 ng/ml fibroblast growth factor-2 (R&D Systems Europe Ltd., Abingdon, U.K.). The phenotype of all donors of MSC was tested by flow cytometry on a FACSCanto II system (Becton Dickinson, Oxford, U.K.) using a human MSC Phenotyping Kit (Miltenyi Biotec, Bisley, U.K.), with positive staining for CD73, CD90, CD105 and negative staining for CD14, CD20, CD34, and CD45 (Supplementary FIG. 9). Experiments were performed using cells at passage 5-7, and all experiments were repeated with cells from 3 donors (21-24 years of age). Donor MSCs were cultured for 72 h with conditioned media collected from 48 h culture of activated human

### HSCs.

The invention is described above and it can easily be appreciated by ones skilled in the art that the methods described allow for improved diagnosis of susceptibility to liver disease, improved diagnosis of susceptibility of a patient to progress from chronic liver disease and the stratification of patient groups into risk categories.

We claim:

1. An in vitro method of determining susceptibility to liver damage in a patient comprising the steps;
providing a tissue sample obtained from a patient;
determining the amount of DNA methylation of the PPAR-γ gene in the sample; and
using the determination to determine the patients susceptibility to liver damage.

2. The method of claim 1 also comprising the step of comparing the amount of DNA methylation of the PPAR-γ gene to predetermined threshold levels.

3. The method of claim 1 also comprising the steps of; determining the amount of DNA methylation of the TGFβ1 gene; and using the determination to determine the patients susceptibility to liver damage.

4. The method of claim 3 also comprising the step of comparing the amount of DNA methylation of the TGFβ1 gene to predetermined threshold levels.

5. The method of claim 1 wherein by comparing methylation levels in a sample to predetermined threshold levels a determination is made as to whether a patient has an elevated risk of progressing from early stage liver disease to cirrhosis.

6. The method of claim 1 wherein the step of determining the level of DNA methylation of the TGFβ1 gene determines the level of methylation of DNA at the TGFβ1 gene promoter.

7. The method of claim 1 where methylation of the PPAR-γ gene of 7% suggests a medium risk, >7% suggests higher risk, >10 suggests high risk, <7% suggests lower risk and <2% low risk.

8. The method of claim 1 which is used to stratify groups of patients into groups susceptible to cirrhosis and those where liver disease is less likely to progress to cirrhosis.

9. The method of claim 1 wherein the step of determining the amount of DNA methylation determines the percentage of CpG methylation.

10. The method of claim 1 wherein pyrosequencing is used to determine methylation levels.

11. The method of claim 1 wherein the method also includes the steps of determining the amount of methylation of the PPAR-α gene.

12. The method of claim 1 wherein the tissue sample is taken from the liver.

13. The method of claim 1 wherein the tissue sample is a blood sample.

14. An in vitro method of determining susceptibility to liver disease in a patient comprising the steps;
providing a tissue sample obtained from a patient;
determining the amount of one or more of PPAR-γ, and TGFβ1 proteins present in the sample; and
using the amount of PPAR-γ, and/or TGFβ1 proteins present in the sample to determine susceptibility to liver disease.

15. The method of claim 14 further comprising the step of comparing PPAR-γ, and/or TGFβ1 protein levels to predetermined threshold levels.

16. The method of any of claim 14 wherein the tissue sample is taken from the liver.

17. The method of any of claim 14 wherein the tissue sample is a blood sample.

18. An in vitro method of determining susceptibility to liver disease in a patient comprising the steps;
providing a tissue sample obtained from a patient;
determining the amount of histone modification of the PPAR-γ gene in the sample;
using the amount of histone modification of the PPAR-γ gene in the sample to determine susceptibility to liver disease.

19. The method of claim 18 wherein the step of determining the amount histone modification of the PPARγ gene identifies the amount of H2A.Z and/or H3K27me3 present.

20. The method of claim 18 further comprising the step of comparing the amount of histone modification to predetermined threshold levels.

21. The method of claim 18 wherein the step of determining the amount histone modification of the PPARγ gene identifies the amount of histone modification of the PPARγ gene promoter.

22. The method of claim 18 wherein the tissue sample is taken from the liver.

23. The method of claim 18 wherein the tissue sample is a blood sample.


SEQUENCE LISTING (4 sequences)

SEQ ID NO: 1 - DNA, 2233 bp, Rattus norvegicus
  exon (614)..(1113)
ggctctgagccgcccgcggaaccggcctccgctgggagccggcaagggagcagcggaggagccgtccgaggccccagagtctgagaccagccgccgccgccgcagggaggagggggaggaggagtgggaggagggacgagctggttgggagaagaggaaaaagttttgagacttttccgctgctactgcaagtcagagacggggggactgcttggcgctgcgctgtcccgcaaggaggcaggacctgaggactccagacagccctgctcaccgtcgtggacactcgattgctacccggcgttcctcggacgcccccattccggaccagccctcgggagccacaagccccgcctcccgcaaagacttcaccccaaagctggggcgcaccccttgcacgccaccctccccccagcctgcttcttgagtcccccgcatcccaggaccctctctcctctgggaggccgatctccctcggacctgctggcaatagcttcctatttaagaacaccccacttttgggtcccagagagcgctcatctcgatttttatcccggtggcatactgagacactctggtgtcagagcgtcaccgcgactcctgctgctttctccctcaacctcaaattattcaggactatcacctacctttccttgggagaccccaccccaccccacaagccctgcaggggcggggcctccgcatcccacctttgcccggggttcgcgctctccgaagttccgtggggcgccgcctcccccatgccgccctcggggctgcggctgctgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccgagccagggggaggtaccgccgggcccgctgcccgaggcggtgctcgctttgtacaacagcacccgcgaccgggtggcaggcgagagcgctgacccggagcccgagcccgaggcggactactacgccaaagaagtcacccgcgtgctaatggtggaccgcaacaacggtgagctctgaggggctggggagccaggagggagcccccagggggcgccggagtgcaggggtcaccgggaggaaattacccgcagaggaaactggctggaggaaggggacccctgggggcgctgggatgactgtttgggtgtcccaaagaggatggcgcttacccctaccccaggtgtctggtcttagagagaaagacagcctgcaaaagtgaacctcccctagaaaggcatgatcctggggagagtccactctagaagaggagcatttgtcaggttttgttttaagttccccttatggtgcctgaaaatgcaaggctcgttgggtccttttccagaaaggtaaagcccggctcaggaaaaatggaaggcgcccagcatttgggaaacaagagacagtgtggaaagccgacactagaccgggtcgtgagatggagagaagagattcaagggaaaaaaaaaacctaggggatgggtaccctagaaatgccaaataaaatctctactgggcttactaccagaaggcgacaggcgcgtgctggagggtgtgatctgtggagtcattgtggggttccaagtgatagaggaaagcgaagttgttaggagaaggaggctagagagagaggagcggagagggtgcggaaaatgagcccggaggtgtcacgagtgttttgcttgggggcgtaaaaaagcttgcgagcgggcaggcggagcccagtggaggaagcgagcgagtcctcaagaaatttagaatactgggtcattgggaaggaattataaaagaggggagactggcgtaggaaggtggccccgcgaggcgggcaggcagaacagggatactatgccatccaggcgagagtggcgagcgcccctcgcctgggcgtggaagggagtggctgaacctcgggccctaggggagctgctagaggtaggagagaggcagtcttcaaaacccaaatgaaaaaaacgagtcggtaagggtgaggaatctccacgtgggtgccacgcgcgtggggaggagcttgcgcttcttgagggggttagaggaccccacggagctgaacgctggcaggctgtacccccaaagtcccagttccttctacgggaggctggggaaaaccc

SEQ ID NO: 2 - DNA, 1417 bp, Rattus norvegicus
  exon (1176)..(1301)
agtcgccccgggccacaaggcccgccgcagggacgctgaagaagagacctggggcgccgcactggggtaccgggtcgtgtgaggcctgaggggactgagtgtgacgacaaggtgaccaggctgaggggacgggctgaggagaagtcatactcagacaggacccggtgagactgacagcctgacgaggtctctctcggctgaggggacgcgggctgagaagtcactttctgaccggactgtgtgacagacaaggtgagggggcctcggcctgtggggactggatctgactgtctaggtgactgggtctcggctgaggggacgcgggatgaggagaagccactctctgtcaggactgtgtaactgacagcctaatcctgtctcaactagaggggacgtgggctggaaagtcgcactctgacaggacggagcgtgacagacaaagtgacggaatcttaactgaggggactgaggagaagtcatactcttgacaggaccaaatgtgacagacaaggtgatcgagtctgggtctgagggggatgcggtctgaggagaagtcacttaggagaagagtgagagagcgtgatagcggggcggggtcggagtgaggggtctcggctgaggaccggtcacccgcctagagagccgagtgtgactgacaaggtgaatgtcctcgtgaggggcacggcccggcaggcccggagggccacagtggagccacctcaggtttcctcagaaggtgctccggcaggcgtgtgcgagctcggagcagggtagcccgggcagcccgaccctgacaggaaagccaggtctccgcacggtccagaactcccgcgggaggccgcattgtgtggatctggtgacagttttcagagaggggctcgccagtagccaccgggtaccccgaggcgctgggggtggggggaggagggtgcgccggtgatcgtctaagcaacttggcccggggctcccgcccgcccggggctcgccaggtgtgaggaaaatggggtcactgtcgccaaggccccgccccaccagtgcccctcttcagcctaggaagggggcgagcacagggcagggctaactggagtaggcagccctcgtggtgggggtggaaggtgggtgtgtggctccggtgctttatggcttgggagtggggaggagcctaaggtgaggaggcagaaactgggagtagcctgggctgcttttatataagggactcgaggaggtcaagaaggggcctggacctctgctggtgatcagaaggctgcagcgctaaattcatcttaactcccattgagtaggtgagtggctgcatcgtcatggcactttgttagagggtgttgtgaagtggccaaactgccccaagttcttacacatcaggatggtttggctgtgttcttgtactgtaagaatctgc

SEQ ID NO: 3 - DNA, 1033 bp, Rattus norvegicus
  exon (734)..(984)
taccagacccgcagccttgaacttcagttctggccggtgcgcggggctgggagcagaaaggcgcgctcgcccaggcgcgcccttcccacagggctgtctcgcccgtcgggtgaccttgggcagtcccttcacctagccagcctcagtttaccaacagatgcccgagccccgaggcactaaatgggcatcgaggagaactgcccagggcctcttaggccccgccctcacagcagcggccaatcagatgctgccgtccggggggtgtgtctcgttctgaaccgcggcccgggcctagagggcggggtttccggggcggtcacctcgccgcgggaccccgcaggggacgtccgaggggcggcgcgtgtcgtgggggcgcggctggcacgggcgcgcgtaggcggtgccgggcaggggccccggacgctgcggccccacgacaggggtgacgggggcggagacagccgctcgagcccctcctggcgccgcctacccgggcgggctggccctgcggacccgcaggcggagcgcagcctcaggtgcccaggggcgggagggcacgcgcgaggacgggagccaggcgtcccccgtcccgggacagtgaggtgggtggacagggaggggaggggctcggtggcgcatgcgcgtggaccaggggcgcgggtctggagacccacagccactggcgagggcacacgctaggaagggcacacgcgtgcgactttcggggcccctggaactgtccgctacttcgagtccccttgagcgccgtgtgccggctctgaacattggcgttcgcagctgttttgtgggctggagggttcgtggagtcctggaactgaagcgacgctgggtcctctggttgtccccttgaggggagggcacacgagcggggacatcggggcgctcccttcccacagcgtggtgcatttgggcgtaactcaccgggaggcgtttcctgagaccctcggggatcttagaggcgaggtaactcgaggccccgggacagactgattctggggtcactgagtctagc

SEQ ID NO: 4 - DNA, 1092 bp, Homo sapiens
  exon (601)..(908)
cagtggtcagtccctgtaagatccgtggctctcagcttcttatcttaggggctgtggaggaaggacatgattatgttgatttaagcgctgaatattttcccttgtgatacccatcctcgcaaaactttgcttcaaccacaaacgaggaccttctgtaccagaggggcaataaccacaatgaagctaggaagaaatgcagagcaccccagcatacagtccataagcttcctgaagtggggggcctcaggcatcgctgcctccccaaagaggatcaggcccagaacagtatgctccagaaataagactggaaaaagggaaagaggggcctcaagtccaggagaccagcggctttctgaacgcgcacctgccaacccactttggacaggtcacgatggacagcgtggcaggaaaagaaaaggtcactgtctacccaacacatgagaaactgtttctcgtgcctcacgtccccactccgtccccacccatgttgtctgagtccctcggtgtcagaaacactgctaagaaatttaagaaattctgttaatgagtttaagaaatgtttttaatgattaaaagtcagtgacttgtgaataaccatgtaacttacaaacgcaaggaactctgaaagtgtgcagcaccaccgatcagaagagaaaaccaagggacccgaaatatgctttaattaaattttcttttaaaatgtcactggaaagaacatcttgggaagacggcctggccgatcgccgtgtgaagggcaagccactctggccgagagggagccccacacctcggtctccccagaccggccctggccgggggcatccccctaaacttcggatccctcctcggaaatgggaccctctctgggccgcctcccagcggtggtggcgaggagcaaacgacaccaggtagcctgccgcggggcagagagtggacgcgggaaagccggtggctcccgccgtgggccctactgtgcgcgggcggcggccgagcccgggccgctccctcccagtcgcgcgccgccgcccccgcccccgcccccgcccccgcccccacccccacccccacccccacccccagccggcgcccgcg